(19) United States
(12) Patent Application Publication
(10) Pub. No.: US 2007/0138316 A1
(43) Pub. Date: Jun. 21, 2007

(54) Fluid dispensing device

(76) Inventors: Rangaswamy G. Iyengar, Highland Park, IL (US); Adam Morrison, Belvedere, IL (US)

Correspondence Address: FAY SHARPE LLP, 1100 SUPERIOR AVENUE, SEVENTH FLOOR, CLEVELAND, OH, 44114, US

(21) Appl. No.: 10/549,895

(22) PCT Filed: Mar. 22, 2004
(86) PCT No.: PCT/US04/08870

§ 371 Date: Aug. 7, 2006

Related U.S. Application Data
(60) Provisional application No. 60/456,315, filed on Mar. 20, 2003.

(51) Int. Cl. A62C 13/62; B05B 7/30; A61M 11/02; B05B 7/04

(52) U.S. Cl. 239/302; 239/369; 239/354; 239/398

(57) ABSTRACT

A fluid dispensing device for dispensing a fluid on an associated surface comprises a dispensing assembly which includes a handle and an outlet for dispensing therefrom compressed air from an associated compressed source. The dispensing assembly selectively dispenses an associated fluid from the associated fluid reservoir with the compressed air dependent on a position of the handle. A passage communicates with the outlet and the associated fluid reservoir. A valve is received in the passage between first and second valve seats. The valve provides selective communication between the associated fluid reservoir and the outlet. A movable stem is operatively associated with the handle and the valve. In a first position, the valve engages the first valve seat to preclude fluid flow from the associated reservoir. In a second position, the valve is interposed between the first and second valve seats for supplying an associated fluid from the associated fluid reservoir to the valve seat. In a third position, the valve engages the second valve seat and precludes fluid flow from the associated reservoir, for example, when pressure increases in the reservoir or if the dispensing assembly is inverted. However, the valve may be displaced from sealing engagement with the second valve seat by depressing the handle which, in turn, moves the stem into engagement with the valve thereby unseating the valve from the second valve seat.

### CROSS REFERENCE TO RELATED APPLICATIONS

This application claims priority from U.S. Provisional Patent Application Ser. No. 60/456,315 filed Mar. 20, 2003 and is incorporated herein by reference.

### BACKGROUND OF THE INVENTION

This application relates to spray devices that are particularly useful for spraying a fluid from a reservoir. For example, paints, insecticides, garden products, lubricants, etc. are representative of the type of fluid or liquid products that may be selectively sprayed with one of these devices.

Various problems exist with known spraying devices. For example, these devices commonly use a supply of compressed air passed through a venturi, the outlet of which is located adjacent a flow passage in communication with a reservoir of a fluid. The venturi draws the fluid from the reservoir where it is mixed with the compressed air and sprayed on to an associated surface.

The venturi outlet and the flow passage are disposed in fixed relation so that the desired suction and withdrawal of fluid from the reservoir, and introduction of the fluid into the compressed air flow are achieved. Some arrangements add a valve to the arrangement to selectively close the flow passage and thereby control the dispensing of the fluid. However, these arrangements still encounter problems with changeover of the fluid, inoperability at various angles (e.g. upside down), fluid leakage, and the like.

Another problem is that portability of the spraying device is a concern since the device must be connected to a remote compressor, power source, etc. Multiple lines, for example, extend from the spray device and are connected to a power source (e.g., A.C. source), compressed air supply, and/or fluid reservoir. A need exists for a portable, self-contained assembly that can be easily manipulated without encountering tangled supply lines or limiting areas of use.

### SUMMARY OF THE INVENTION

The present invention discloses an apparatus and method for dispensing a fluid on an associated surface.

The apparatus comprises a dispensing assembly which includes a handle and an outlet for dispensing therefrom compressed air from an associated compressed source and selectively dispensing an associated fluid from an associated fluid reservoir with the compressed air dependent on a position of the handle. A passage communicates with the outlet and the associated fluid reservoir. A valve is received in the passage between first and second valve seats. The valve provides selective communication between the associated fluid reservoir and the outlet.

A movable stem is operatively associated with the handle and the valve. In a first position, the valve engages the first valve seat to preclude fluid flow from the associated reservoir. In a second position, the valve is interposed between the first and second valve seats for supplying an associated fluid from the associated fluid reservoir to the valve seat. In a third position, the valve engages the second valve seat and precludes fluid flow from the associated reservoir.

In another embodiment, the apparatus comprises a fluid reservoir adapted to store an associated fluid therein, a dispensing head and a spray head. The dispensing head has a first passage that communicates with the fluid reservoir at one end and includes a valve received therein between first and second valve seats to provide selective dispensing of fluid from the fluid reservoir. The spray head is operatively associated with the dispensing head and includes a second passage that communicates with an associated source of compressed air and with an air dispensing opening. A means is provided for selectively positioning the valve to direct fluid from the fluid reservoir towards the air dispensing opening and for sealing the first passage in response to increased pressure in the fluid reservoir.

In another embodiment, the apparatus comprises a housing defining an internal cavity adapted to receive a portable power source and a compressor assembly. The compressor assembly is selectively operated by the portable power supply. Means is provided for connecting the housing to an associated fluid reservoir that stores a fluid to be dispensed on an associated surface.

A primary benefit of the invention resides in the ability to operate the spraying device at various angles.

Another benefit of the invention resides in the portability of the spraying device that does not need to be connected to a remote compressor and/or power source.

Still another benefit of the invention is that the spraying device is a self-contained assembly that can be easily manipulated without encountering tangled supply lines or limiting areas of use.

Still other benefits and advantages of the invention will become apparent to those skilled in the art upon reading and understanding the following detailed description.

### BRIEF DESCRIPTION OF THE DRAWINGS

FIG. 1 is an exploded view of a first embodiment of the invention.

FIG. 2 is a longitudinal, cross-sectional view of a second embodiment of the invention.

FIG. 3 is a perspective, cross-sectional view of a second embodiment of the invention.

FIG. 4 is a longitudinal, cross-sectional view of a second embodiment of the invention.

FIG. 5 is a longitudinal, cross-sectional view of a second embodiment of the invention in a first position of the stem, valve and valve seat(s).

FIG. 6 is a perspective, cross-sectional view of a second embodiment of the invention in a first position of the stem, valve and valve seat(s).

FIG. 7 is a longitudinal, cross-sectional view of a second embodiment of the invention in a second position of the stem, valve and valve seat(s).

FIG. 8 is a perspective, cross-sectional view of a second embodiment of the invention in a second position of the stem, valve and valve seat(s).

FIG. 9 is a longitudinal, cross-sectional view of a second embodiment of the invention drawing the fluid contents from an associated fluid reservoir and mixing the fluid with pressurized air in a spraying operation.

FIG. 10 is a perspective, cross-sectional view of a second embodiment of the invention drawing the fluid contents from an associated fluid reservoir and mixing the fluid with pressurized air in a spraying operation.

FIG. 11 is a perspective, cross-sectional view of a second embodiment of the invention wherein the valve is engaged with the valve seat and the fluid head is in a lower position.

FIG. 12 is a longitudinal, cross-sectional view of a second embodiment of the invention in a third position of the stem, valve and valve seat(s).

FIG. 13 is a perspective, cross-sectional view of a second embodiment of the invention in a third position of the stem, valve and valve seat(s).

### DETAILED DESCRIPTION OF THE INVENTION

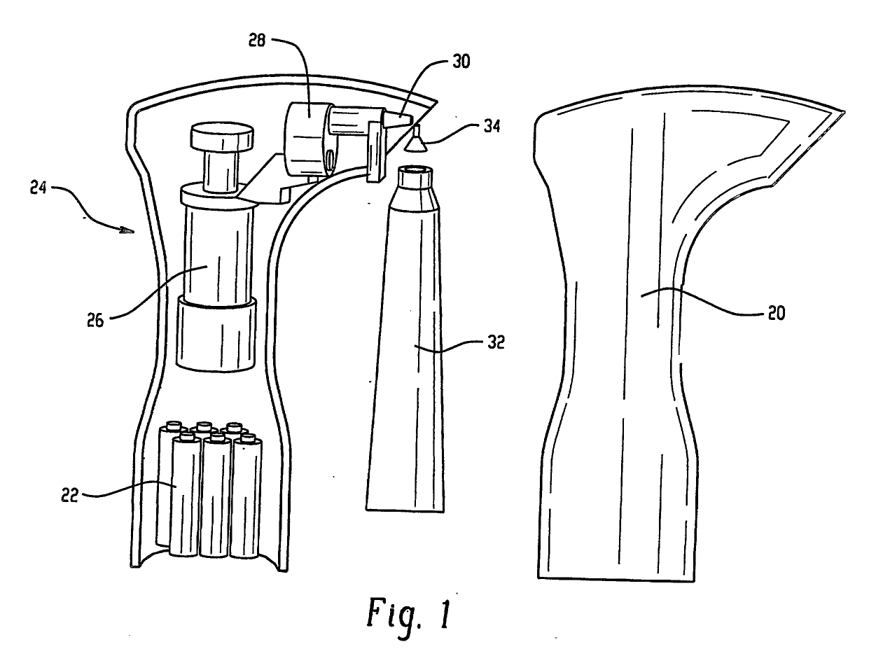

In one arrangement, a molded, two-part housing is snapped together to provide a convenient hand-held unit that includes all of the components necessary for spraying. For example, as shown in FIG. 1, housing 20 is illustrated in blue as a two-part housing assembly, the housing halves being essentially mirror images of one another that may be snap-fit or adhesively secured together once the internal components are assembled therein. Typically, the housing is a polymer structure, although as will be appreciated in accordance with the present invention, alternative materials including metallic materials such as aluminum can be used to form the housing. Thus, housing portions 20a and 20b are brought into mating engagement and define an internal cavity that houses the various components of the hand held sprayer. For example, a power source such as a pack of batteries 22 is provided in a base of the elongated housing. A portable power source is desired in this arrangement so that there is no need for electrical cords during operation. A conventional ON/OFF switch (not shown) selectively connects the power source to a compressor assembly 24 that includes an electric motor 26 that drives an air pump or compressor 28. Specifically, motor 26 is disposed adjacent the power source so that an ON/OFF switch (not shown), selectively energizes the motor to drive the air pump 28. The air pump receives ambient air from the rear of the housing, or up through the base of the handle and around the motor, where the air is pressurized/compressed in the pump and exits through a reduced diameter nozzle or venturi 30. The nozzle is directed outwardly from the housing and passes over a fluid reservoir, such as flexible pouch 32 that stores a fluid to be dispensed on an associated surface.

Dispensing tip 34 extends from the outlet of the fluid pouch and an outlet thereof is located adjacent the nozzle 30 so that as the pressurized air from the pump passes thereover, the venturi creates a negative pressure or suction to draw the fluid from the pouch 32. Thus, a convenient, hand held, gun-shaped spray device is provided that has its own self-contained compressor, power source, and receives the fluid to be sprayed onto the associated surface.

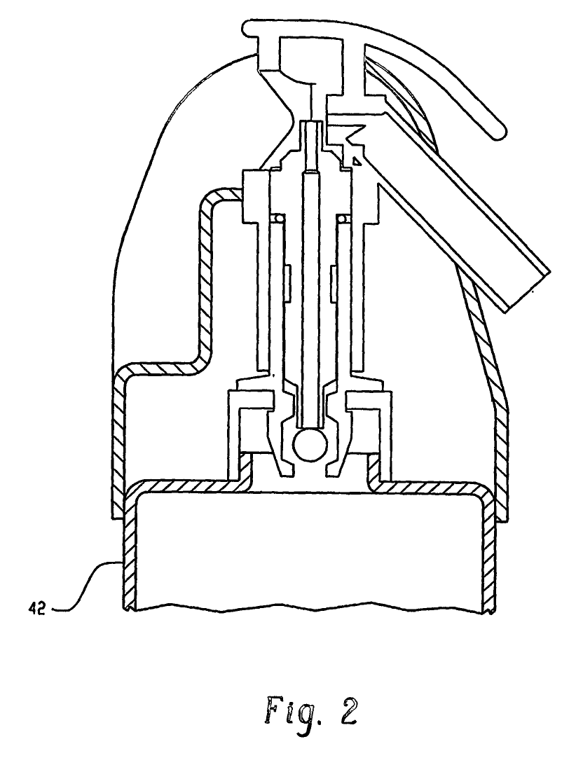

Another spray dispenser device or spraying device is shown in FIGS. 2-13. More particularly, and with specific reference to FIGS. 2-4, connector housing 40 is adapted for connection to an associated fluid reservoir 42. For example, connector housing may be a one piece portion of the associated fluid reservoir or a separable component that connects the fluid reservoir to the spraying device. Extending outwardly from the connector housing is a dispensing head or dispensing assembly 44, which is a generally cylindrical, hollow structure. The dispensing head includes a first end 46 that communicates with the fluid reservoir and has a valve such as ball member 48 received therein. The ball is free to float between a first or upper valve seat 50 and a second or lower valve seat 52. If the ball engages either the first or second seat, then fluid flow from the reservoir is precluded. As shown, flange 54 of the dispensing head cooperates with flange 56 of the connector housing to secure the dispensing head to the connector housing in fixed relation.

Received around the dispensing head is a spray head or housing 60. The spray head includes an elongated, generally cylindrical portion or outer sleeve 62 concentrically received around cylinder portion 44 of the dispensing head. The spray head includes a handle 64 extending outwardly at an upper end thereof to allow for selective depressing movement of the spray head relative to the housing and dispensing head. The spray head further includes a passage 66 that communicates (as represented by dashed line 68) with an external compressor represented by box 70. For example, the dashed line may be a conventional air line or passage 68 of extended length, e.g., eight to twenty (8-20) feet, extending from a conventional air compressor 70 to provide a flow of pressurized air to passage 66 of the spray device. The compressed air passes through a smaller diameter passage 72 that extends generally perpendicular to the longitudinal axes of the dispensing head and spray head. The air dispensing opening 72 is provided with a constant supply of pressurized air from the compressor and thus, when air is supplied to line 68, the spray device emits pressurized air. By selectively depressing handle 64, fluid is selectively drawn from the fluid reservoir 42 for mixing with the airflow and spraying or dispensing atomized fluid on to an associated surface as will be described further below.

As will be appreciated by those of ordinary skill in the relevant art, as an alternate to the aforementioned conventional air compressor assembly, the spray head assembly further includes a separate housing (not shown) attached underneath the handle. The housing has first and second mating portions which define an internal cavity that is adapted to house an associated compressor assembly and power source such as one or more batteries. A portable power source is desired in this arrangement so that there is no need for electrical cords during operation. A conventional ON/OFF switch (not shown) selectively connects the power source to a compressor assembly that includes an electric motor that drives a compressor. Specifically, a motor is disposed adjacent the power source so that an ON/OFF switch (not shown), selectively energizes the motor to drive a compressor. The compressor receives ambient air where the air is pressurized/compressed in the pump and exits through the smaller diameter passage 72. As such, the spray head emits pressurized air.

The dispensing head 44, as noted above is hollow, and includes an internal passage 80 that receives a fluid or liquid head assembly 82 also referred to as a movable, hollow stem. The fluid head assembly is preferably a generally elongated, hollow cylindrical member. Internal passage 84 extends through the fluid head and terminates in a first or bottom opening at one end 86, and a second or upper opening at the other end 88. The fluid head 82 is adapted for sliding movement relative to the dispensing head and the spray head. The first end 86 selectively engages ball 48 to urge the ball away from the upper seat 50 of the dispensing head. This allows fluid from the reservoir 42 to pass by the lower valve seat, around the ball, and into the passage 84 of the fluid head where the fluid is mixed with the compressed air from air opening 72. Accordingly, it will be appreciated that when the end 86 of the fluid head engages the ball, the dimensional contours of these engaging components allow fluid to enter the passage 84. Typically, the housing; dispensing head assembly; spray head assembly; and liquid head assembly are polymer structures, although as will be appreciated in accordance with the present invention, alternative materials including metallic materials such as aluminum can be used to form same.

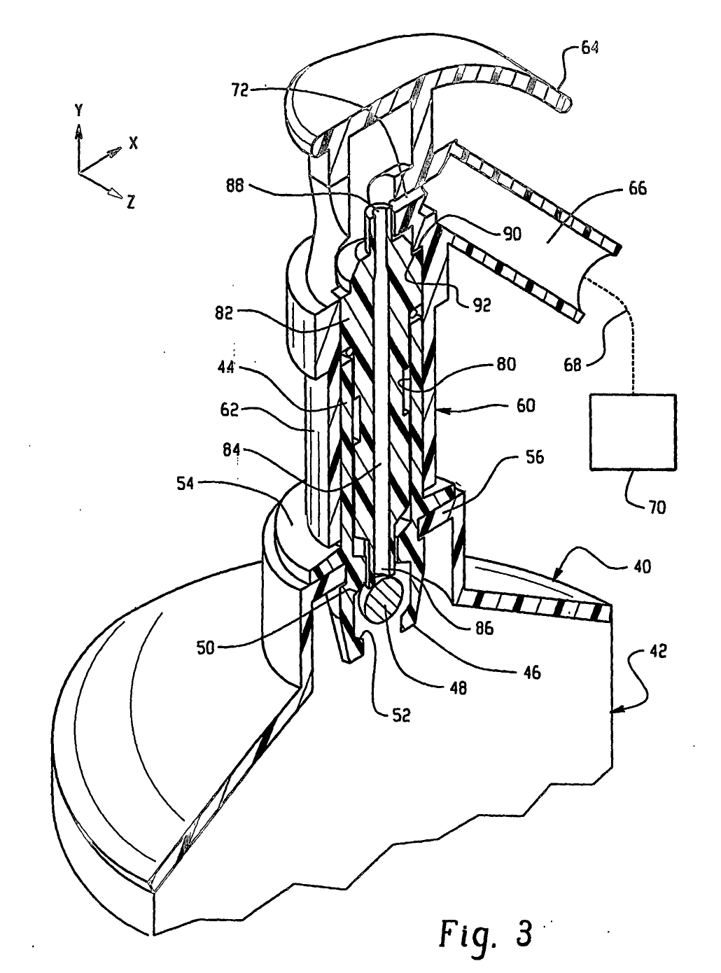

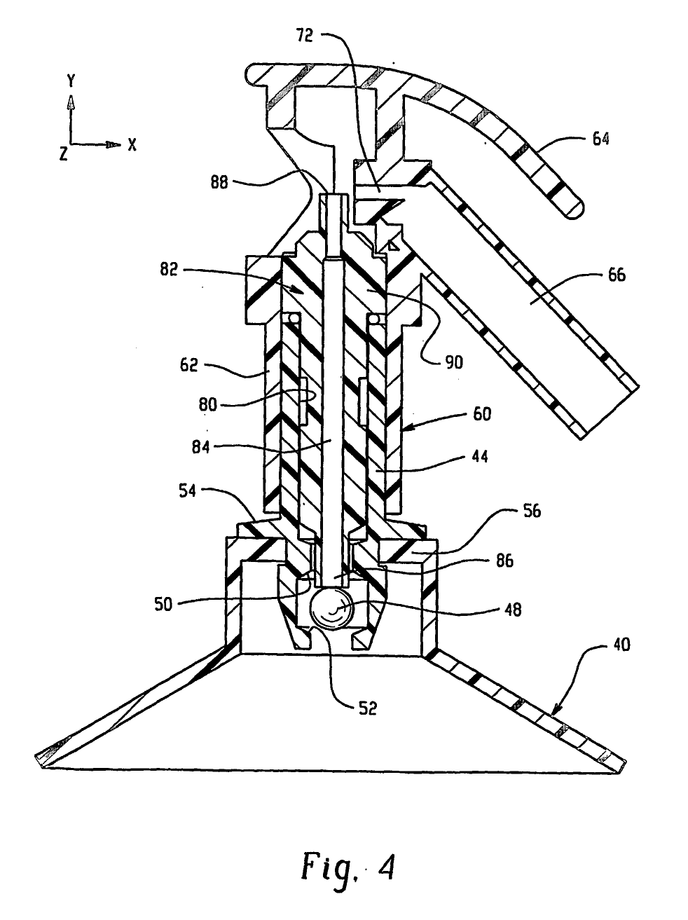

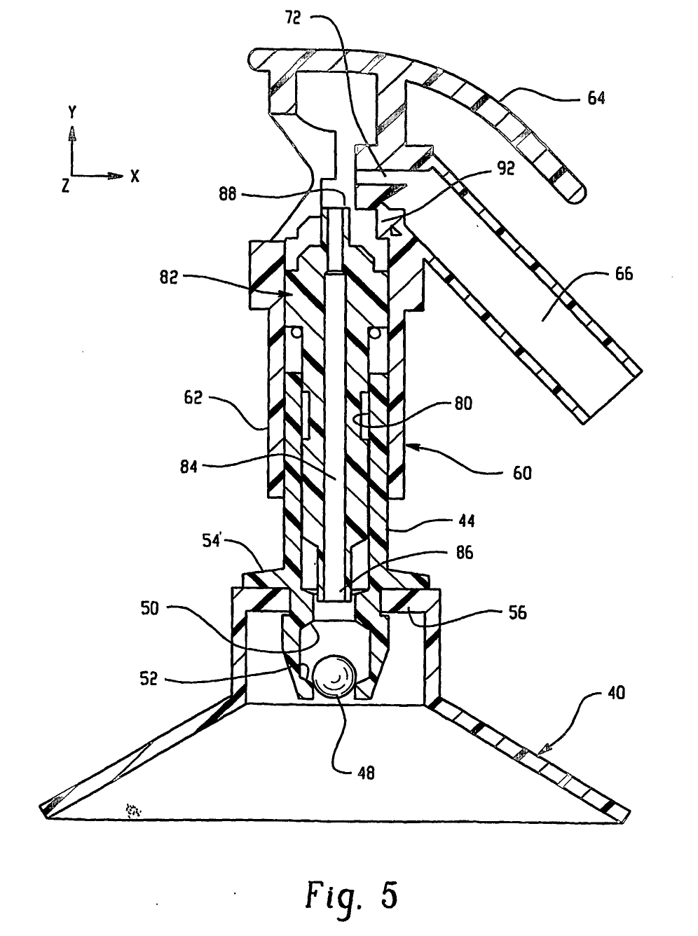

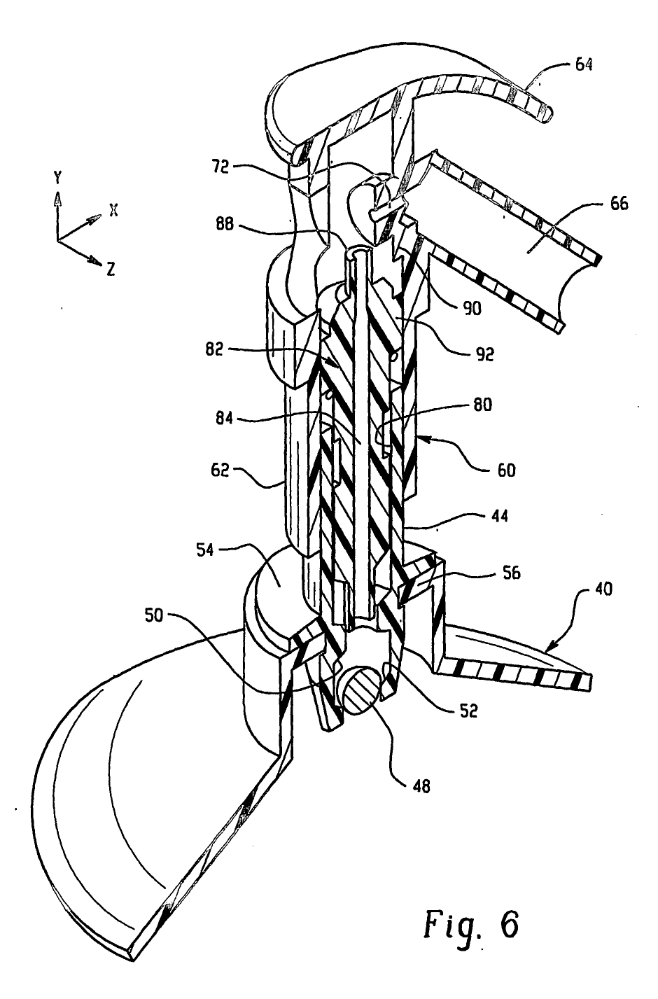

The relationship of the components of the spray device in FIGS. 3 and 4 should be contrasted with that in FIGS. 5 and 6. In the latter, the ball is engaged with the lower seat 50 to preclude the passage of any fluid from the reservoir 42. Only compressed air through opening 72 is dispensed from the spraying device. The spray head is located so that its upper end 88 is spaced from or below the air passage 72. This gap or spacing between the end opening 88 and the air opening 72 is sufficient to prevent a sufficient vacuum or suction force to be developed through the liquid head. Thus, the elongated passage 84 of the liquid head does not introduce sufficient vacuum to draw the ball off of the lower seat or draw fluid from the reservoir in a first position of the stem, valve and valve seat(s).

As shown in FIGS. 5 and 6, the ball is seated. This illustrates that fluid previously removed from the reservoir partially collapses the wall of the reservoir and imparts a backpressure that draws the ball into engagement with the lower seat. This prevents introduction of air into the reservoir when spraying is terminated and, in fact, the wall of the reservoir remains partially collapsed. Such action is desirable since the next time that fluid is to be sprayed, fluid is immediately introduced into the passage 84. Without this feature, air must be removed from the fluid reservoir and impacts on the amount of fluid drawn into the passage, i.e., the fluid and air from the reservoir will both be drawn into the passage 84 rather than just the desired fluid.

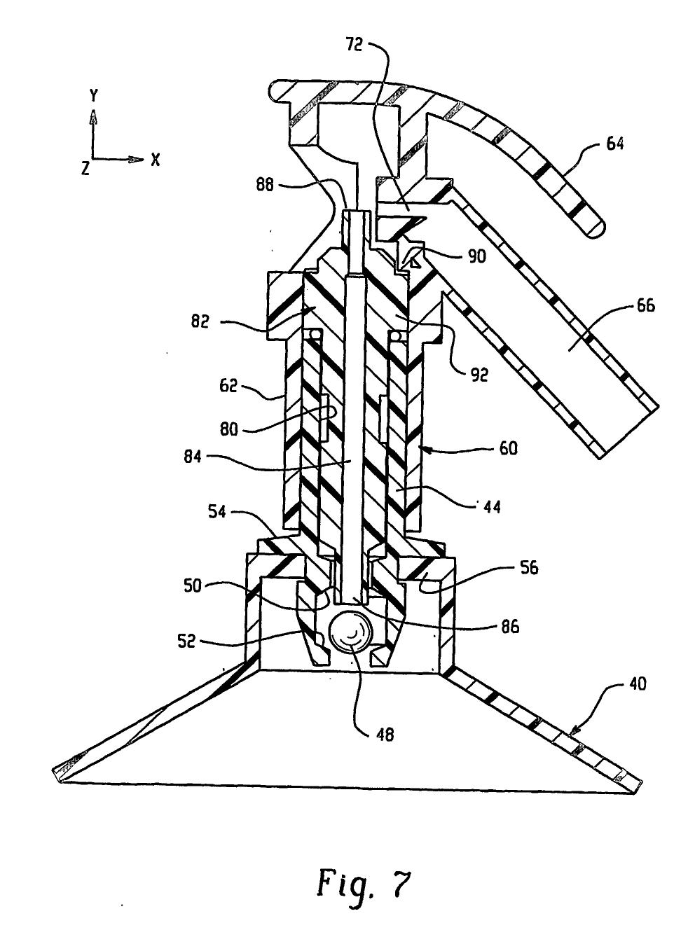

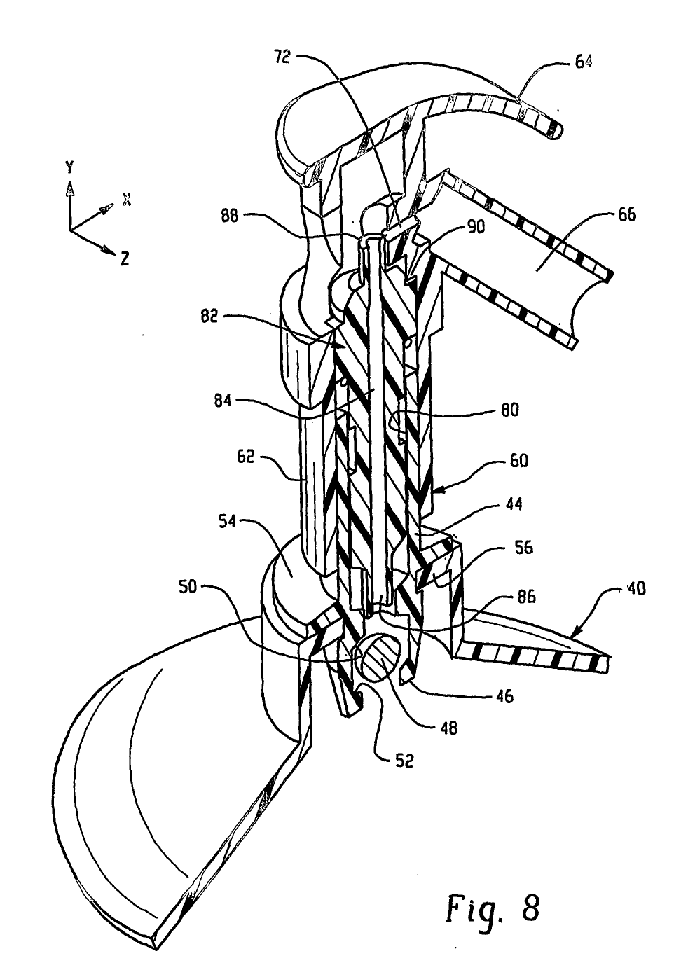

Turning now to FIGS. 7 through 11, pressing down on handle 64 allows cooperating shoulders 90 on the spray head and 92 on the fluid head to selectively engage one another and urge the liquid head downwardly into the passage 80. The depressing action on handle 64 also brings the air opening 72 into alignment with the end 88. Thus, the high pressure airflow passes across the end 88 develops a suction force to draw the fluid contents from reservoir 42 and around the ball 48. The lower end 86 of the stem of the liquid head assures that the ball does not engage the upper seat. Thus, effective spraying is achieved in a second position of the stem, valve and valve seat(s) as illustrated in FIGS. 7 and 8.

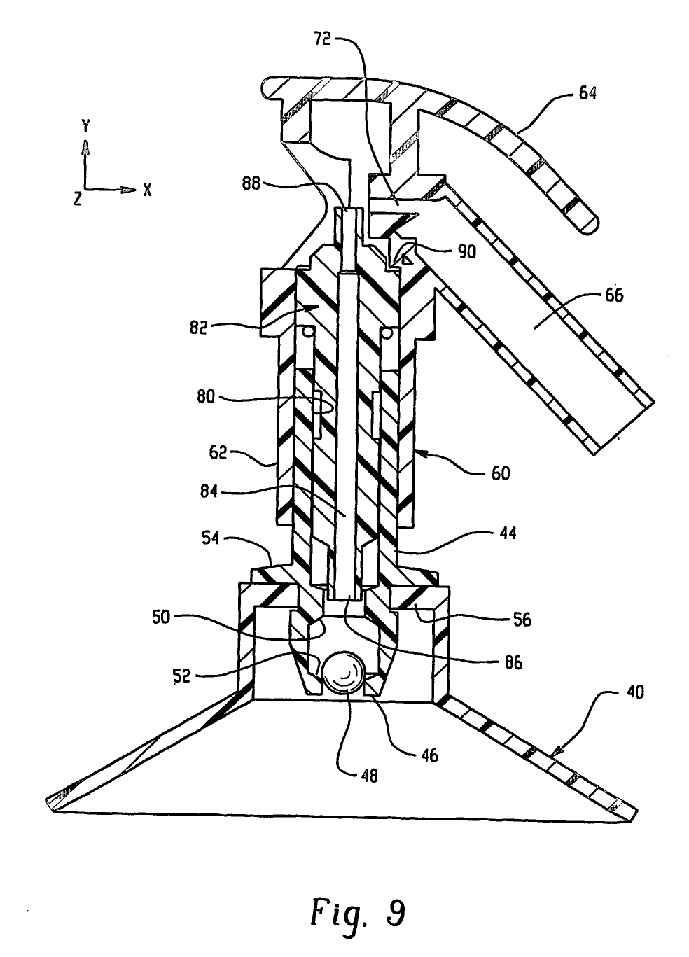

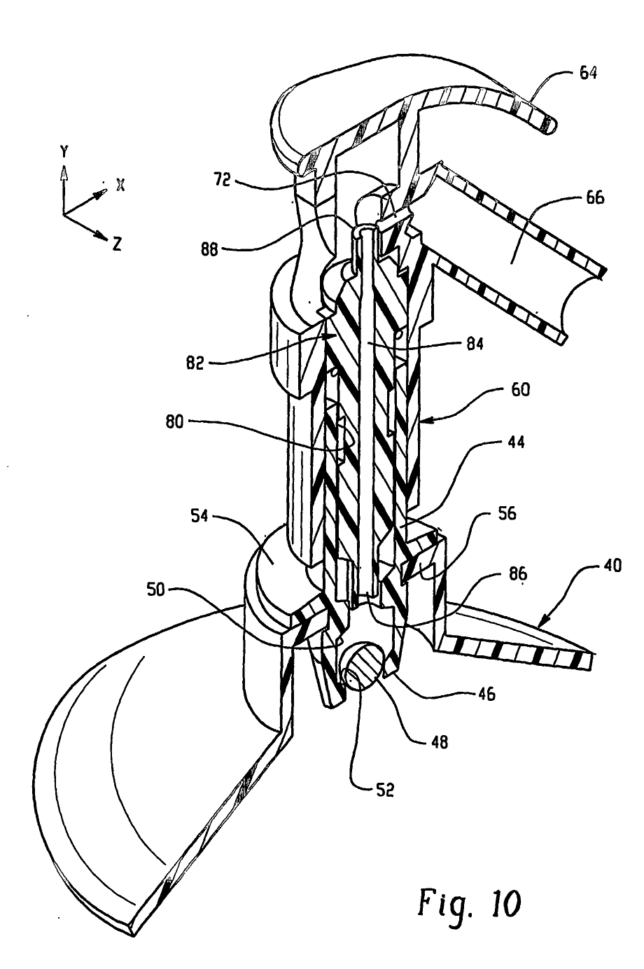

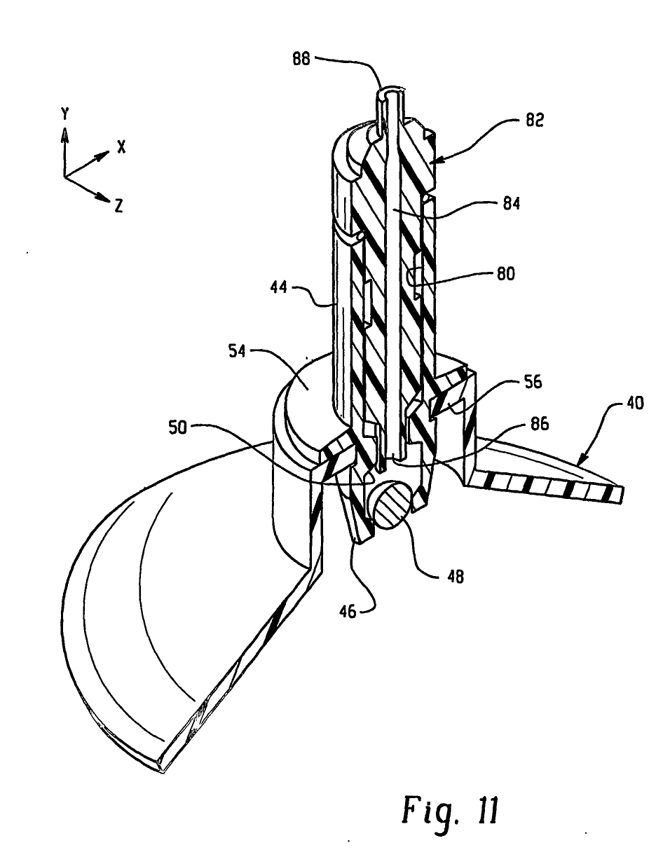

FIGS. 9 and 10 illustrate the sprayer device drawing the fluid contents from the reservoir and mixing the fluid with pressurized air in a spraying operation. The fluid head is positioned so that end 88 is adjacent the air opening 72. The fluid head is not engaging the ball in this illustration to represent that the ball can be positioned at various locations between the upper and lower valve seats. In FIG. 11, the ball member is engaged with the seat and the fluid head is shown in a lower position.

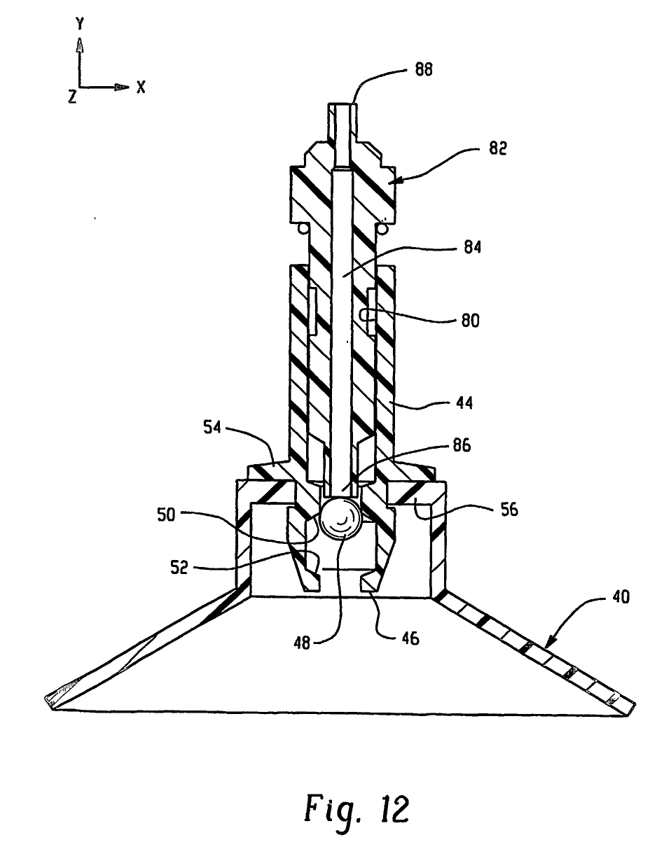

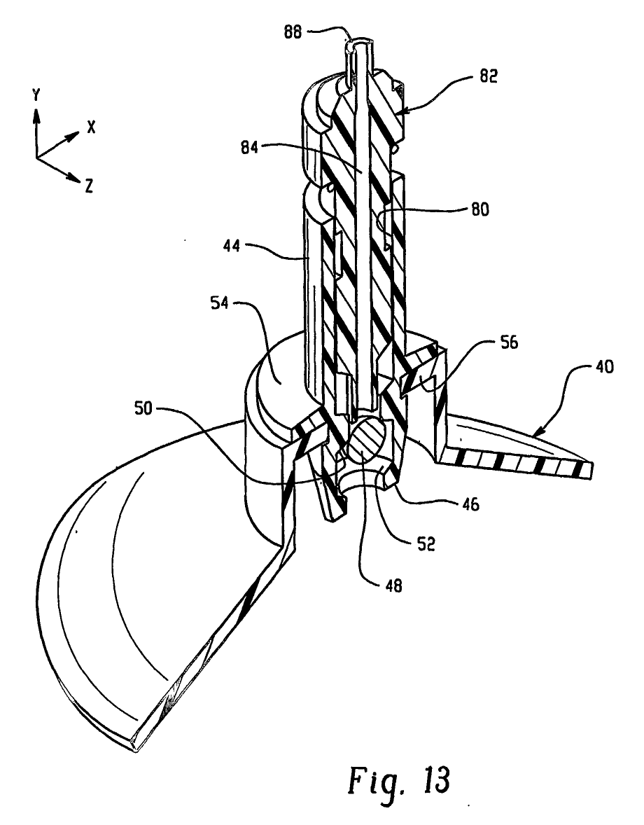

If the reservoir is compressed or squeezed, the ball moves to the position shown in FIGS. 12 and 13 where it engages the upper valve seat and precludes spilling of the fluid contents from the reservoir in a third position of the stem, valve and valve seat(s). The ball moves upwardly and if the fluid head is in a lower position, the fluid head is also urged to the upper position shown in the figure once the ball engages the lower seat. For example, it is contemplated that one preferred form of fluid reservoir will be a flexible wall or pouch that stores paint for instance. Without the provision of the second or upper valve seat, fluid from the reservoir could pass around the ball and be inadvertently spilled through passage 84 when pressure increases in the reservoir as a result of squeezing the paint pouch, or as a result of increased reservoir pressure due to other circumstances. Likewise, if the spray device is inverted or turned upside down, the contents of the fluid reservoir could inadvertently leak through passage 84. However, the ball will seat against the upper valve seat in such a circumstance.

It will be appreciated that the entire spray device can be moved upside down, right side up, or at any angle therebetween, and still effectively spray the fluid contents of reservoir 42. To achieve this, the relative movement between air opening 72 and the opening 88 in the liquid head determines whether or not only compressed air is being dispensed or whether the dispensed air is drawing fluid from the reservoir. More particularly, as noted above the ball engages the upper valve seat 50 when the spray device is turned upside down. However, if the handle is actuated/depressed, then the first end 86 of the fluid head engages the ball 48 and moves the ball from seated engagement with the upper valve seat. In this manner, fluid (such as paint) can be dispensed from the spray device even when the spray device is turned upside down.

Still another important feature is that once the handle 64 is released and the air opening 72 separated from opening 88 in the liquid head, no air is drawn into the reservoir. The ball engaging the lower seat achieves this closing action and this occurs, again, irrespective of the orientation of the spray device.

The invention has been described with reference to the preferred embodiments. Obviously, modifications and alterations will occur to others upon reading and understanding the preceding detailed description. It is intended that the invention be construed as including all such modifications and alterations.

1. A spraying device adapted for connection with an associated fluid reservoir, the spraying device comprising:
a dispensing assembly having a handle and an outlet for dispensing therefrom at least compressed air from an associated compressed source, and selectively dispensing an associated fluid from the associated fluid reservoir with the associated compressed air dependent on a position of the handle;
a passage communicating with the outlet and the associated fluid reservoir;
a valve received in the passage between first and second valve seats, the valve providing selective communication between the associated fluid reservoir and the outlet; and,
a movable stem that is operatively associated with the handle and the valve such that in a first position, the valve engages the first valve seat to preclude fluid flow from the associated reservoir, in a second position is interposed between the first and second valve seats for supplying an associated fluid from the associated fluid reservoir to the valve seat, and in a third position the valve engages the second valve seat and precludes fluid flow from the associated reservoir.

2. The invention of claim 1 wherein the valve is a ball member received between the first and second valve seats.

3. The invention of claim 1 wherein the moveable stem is an elongated, hollow member.

4. The invention of claim 3 wherein the moveable stem is adapted for sliding movement relative to the dispensing head.

5. The invention of claim 3 wherein a first end of the stem is located adjacent the outlet.

6. The invention of claim 5 wherein a first end of the stem is located closest to the outlet in the second position.

7. The invention of claim 3 wherein a second end of the stem operatively and selectively engages the valve.

8. The invention of claim 1 wherein the movable stem includes an internal passage which extends through the movable stem and terminates in a first opening at a first end and a second opening at a second end.

9. The invention of claim 1 wherein the handle extends outwardly from the dispensing head for selective movement relative thereto.

10. The invention of claim 1 wherein the outlet communicates with an associated compressor assembly.

11. A spraying device comprising:
a fluid reservoir adapted to store an associated fluid therein;
a dispensing head having a first passage that communicates with the fluid reservoir at one end and including a valve received therein between first and second valve seats to provide selective dispensing of fluid from the fluid reservoir;
a spray head operatively associated with the dispensing head including a second passage that communicates with an associated source of compressed air and with an air dispensing opening; and,
means for selectively positioning the valve to direct fluid from the fluid reservoir towards the air dispensing opening to seal the first passage in response to increased pressure in the fluid reservoir.

12. The invention of claim 11 wherein the valve positioning means seals the first passage in response to inverting the dispensing head.

13. The invention of claim 11 wherein the valve is a ball member received between the first and second valve seats.

14. The invention of claim 11 wherein the second passage is an elongated, hollow moveable stem having a first end located adjacent the air dispensing opening and a second end that selectively engages the valve.

15. A spraying device comprising:
a housing defining an internal cavity;
a portable power source received in the housing cavity;
a compressor assembly received in the housing cavity and selectively operated by the portable power supply; and,
means for connecting the housing to an associated fluid reservoir that stores a fluid to be dispensed on an associated surface.

16. The invention of claim 15 wherein the housing includes first and second housing portions that are secured together.

17. The invention of claim 15 wherein the compressor assembly includes a motor operatively connected to means for pressurizing ambient air, and a nozzle which is directed outwardly from the housing.

18. The invention of claim 17 wherein the ambient air pressurizing means pumps the pressurized air through the nozzle over the associated fluid reservoir.

19. The invention of claim 15 wherein the compressor assembly includes a switch secured to the housing.

20. The invention of claim 15 wherein the housing connecting means includes a flange disposed adjacent the nozzle, the flange including an aperture that communicates with the flow path of the pressurized ambient air.
